(19) United States
(12) Patent Application Publication
(10) Pub. No.: US 2022/0236295 A1
(43) Pub. Date: Jul. 28, 2022

(54) PROCESSING CARTRIDGE FOR PORTABLE DRUG TESTING SYSTEM

(71) Applicant: BuzzKill Labs, Inc., Livermore, CA (US)

(72) Inventors: George FARQUAR, Livermore, CA (US); Marcus TOFANELLI, Livermore, CA (US)

(21) Appl. No.: 17/610,574

(22) PCT Filed: May 13, 2020
(86) PCT No.: PCT/US20/32602

§ 371 Date: Nov. 11, 2021

Related U.S. Application Data
(60) Provisional application No. 62/847,140, filed on May 13, 2019.

(51) Int. Cl. G01N 33/94; G01N 33/52; B01L 3/00; B01J 20/08; B01J 20/10; B01J 20/26; B01J 20/28

(52) U.S. Cl. CPC G01N 33/948; G01N 33/526; B01L 3/5023; B01J 20/08; B01J 20/103; B01L 2200/04; B01J 20/28052; B01J 20/28004; B01L 2300/0681; B01L 2300/069; B01L 2400/0666; B01J 20/261

(57) ABSTRACT

A cartridge for sample preparation includes an inlet configured to receive a collected sample, a phase transfer assembly including a plurality of bead layers, and an outlet. The collected sample is configured to be transferred through the bead layers of the phase transfer assembly to extract a compound therefrom. The outlet is configured to transfer the extracted compound to a detector for analysis of the extracted compound.

### REFERENCE TO RELATED APPLICATIONS

This application claims the benefit of the priority date of may be related to U.S. Provisional Application No.: 62/847140, filed May 13, 2019, the entirety of which is incorporated by reference herein.

### STATEMENT AS TO FEDERALLY SPONSORED RESEARCH

None.

### THE NAMES OF THE PARTIES TO A JOINT RESEARCH AGREEMENT

None.

### SEQUENCE LISTING

None.

### BACKGROUND

According to an April 2017 poll, approximately 22% of American adults, or ˜54.5 million people, currently use marijuana, with 63% of this group indicating regular use. There are thus nearly as many marijuana users as there are cigarette smokers (˜59 million cigarette smokers). This number is expected to increase as marijuana legalization becomes more common. Additionally, the Department of Justice reports that the overall availability of controlled prescription and illegal drugs in the U.S. is also increasing or remaining stable at high rates. In a 2017 National Drug Threat report, the DEA noted that more individuals report current use of controlled prescription drugs than for cocaine, heroin, and methamphetamine combined, making controlled prescription drug use second only to marijuana.

As a result of increased drug usage, there are more drug-impaired drivers on the road than ever before. Indeed, drivers in fatal crashes are now more likely to be under the influence of drugs than alcohol. In Colorado, one of the first states to legalize recreational marijuana (2012), a November 2017 survey conducted by the Colorado Department of Transportation found that 55% of marijuana users believed it is safe to drive while under the influence. However, studies show that under the influence of THC, the psychoactive compound in marijuana, a user's reaction time and perception of distance and speed are both impaired.

There is currently not an efficient and reliable quantitative portable test for marijuana and other drug use. In particular, it is currently very difficult to enforce Driving Under the Influence of Drug (DUID) laws with existing oral fluid (i.e. saliva), breath, and blood tests. The lack of adequate roadside drug testing and effective DUID enforcement can result in a larger number of serious and/or fatal accidents. FIG. 1 shows the current testing method, which requires that a Drug Recognition Expert (DRE) be called to the scene to perform a standard 12-step evaluation for impairment. Based upon the results of the DRE evaluation, the officer can determine whether probable cause has been established and arrest the driver. Once arrested and transported to the station, blood tests can be performed and the results sent to the lab for evidentiary purposes. However, the use of Drug Recognition Experts (DREs) to establish probable cause is inefficient because: (a) it requires a trained expert, usually in addition to the detaining officer, doubling the manpower necessary, (b) it takes time for the expert to get to the scene once called, allowing drug levels to drop as drugs are metabolized, and (c) the DRE's subjective assessment still usually requires scientific evidence of drug presence through urinalysis or blood analysis by toxicologists to prosecute. Moreover, blood drawn hours later is not representative of the level of drugs present at the time of the traffic stop. Unlike alcohol, where the elimination rate is well understood, THC and other drug elimination rates are not consistent. Toxicologists therefore cannot perform retrograde extrapolation to estimate the amount of drugs present in a person's blood at a specific time in the past.

In addition to roadside testing, there has been a corresponding increase in need for drug testing in the workplace, police stations, hospitals, and other locations. The most common method currently used for workplace drug testing, for example, requires a urine sample to be collected and sent off to a lab. The sample is then sealed sent to a laboratory for analysis. The resulting test results are then sent to the employer. The results have two significant problems for employers. First, there is often extensive time delay from sample to the results. Second, urine does not accurately reflect the current level of drugs in a person's system. Rather, urine indicates the level of drugs in person at the time the urine was collected in the bladder. If an individual had a full bladder and proceeded to consume marijuana or other drugs, the levels of drugs in the urine would not increase until the bladder was emptied and refiled, yielding a false negative. Similar urine tests are often used in police stations and other medical settings. Accordingly, employers, emergency room workers, and others need a system to rapidly identify the concentration in near real time to aid in identifying if an employee is fit to continue work or to access the concentration of chemical in the sample. FIG. 2 shows an exemplary current urine or blood testing method for workplace and medical settings. The current methods take time for sample transport and require a skilled worker and traditional laboratory equipment.

Accordingly, an efficient and reliable quantitative simple and/or portable test for marijuana and other drugs is desired.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a standard roadside drug testing method.

FIG. 2 shows a standard urine or blood testing method for workplace and medical settings.

FIG. 3 shows a method as described herein for testing for impairing substances.

FIG. 4 shows a method as described herein for testing for impairing substances.

FIG. 5 shows an exemplary system for testing for impairing substances.

FIG. 6A is a diagram of an exemplary cartridge.

FIGS. 6B-6C show exemplary phase transfer interface assemblies.

FIG. 7 shows an exemplary method of gathering a sample and analyzing for a specified compound.

FIG. 8 shows an exemplary base of a cartridge of this disclosure including top-down view (left) and bottom up view (right).

FIG. 9 shows top-down views of an exemplary cover (left) and a base (right) of a cartridge of this disclosure.

FIG. 10 shows an exemplary assembled cartridge (left) and bottom up view of a cover of a cartridge of this disclosure.

FIG. 11 shows an exemplary instrument configured to engage cartridge of this disclosure.

FIG. 12 shows an exemplary system comprising a cartridge engaged with an instrument.

### SUMMARY

In general, in one embodiment, a cartridge for sample preparation includes an inlet configured to receive a collected sample, a phase transfer assembly including a plurality of bead layers, and an outlet. The collected sample is configured to be transferred through the bead layers of the phase transfer assembly to extract a compound therefrom. The outlet is configured to transfer the extracted compound to a detector for analysis of the extracted compound.

This and other embodiments can include one or more of the following features. The extracted compound can be THC. The cartridge can be disposable. The cartridge can further include one or more registration guides configured to mate with a pumping system or the detector. The cartridge can further include one or more reservoirs configured to store the collected sample. The cartridge can further include one or more reservoirs configured to store the extracted compound. The cartridge can further include a second phase transfer assembly configured to extract a second compound from the collected sample. The second phase transfer assembly can include a plurality of bead layers. The plurality of bead layers can include a first layer, a second layer, and a third layer. The first layer can include a filter layer, the second layer can include a selective binding layer, and the third layer can include a phase transfer layer. The plurality of bead layers can include a first layer. The first layer can include a filter layer. The filter layer can include beads between 40 and 600 microns in diameter. The filter layer can include beads with 20% or less disparity in diameter. The filter layer can include inert beads. The inert beads can include polystyrene, sand, or quartz sand. The filter layer can include chemically active beads. The chemically active beads can include beads with a C-18 coating, a chiral coating, a derivatizing or chemical modification agent, or a biological active compound. The plurality of bead layers can include a second layer. The second layer can include a selective binding layer. The selective binding layer can include beads comprising a desiccant. The selective binding layer can include beads comprising a thiol, COOH, NH3, or CHO. The plurality of bead layers can include a third layer. The third layer can include a phase transfer layer. The phase transfer layer can include beads that are configured to reversibly bind compounds. The beads of the phase transfer layer can include quartz, alumina, polystyrene, or silica. The beads of the phase transfer layer can include beads functionalized with C-18, NH3, or COOH. The beads of the phase transfer layer can provide a surface area to volume ratio of at least 500,000 or greater. The plurality of beads layers can include a first filter layer and a second filter layer. The first filter layer can have beads of higher diameter than beads of the second filter layer.

### DETAILED DESCRIPTION

### I. Cartridge

Described herein are portable drug testing apparatuses, systems, and methods that can be efficiently and reliably used to collect evidentiary fluid samples, extract compounds of interest, and/or analyze samples to provide accurate quantitative results. The apparatuses, systems, and methods described herein can be performed on-site (e.g., on the roadside), can be fully automated, and can provide quantitative results for a variety of different compounds or substances.

The compound of interest (analyte) can be, for example, cocaine, opioid, ayahuasca, central nervous system depressant, DMT, GHB, hallucinogen, heroin, ketamine, KHAT, LSD, MDMA (ecstasy/molly), mescaline (peyote), methamphetamine, over-the-counter medicines—dextromethorphan (DXM), over-the-counter medicines—loperamide, PCP, prescription, prescription stimulant, psilocybin, Rohypnol® (flunitrazepam), salvia, steroids (anabolic), synthetic cannabinoid, synthetic cathinone (bath salts).

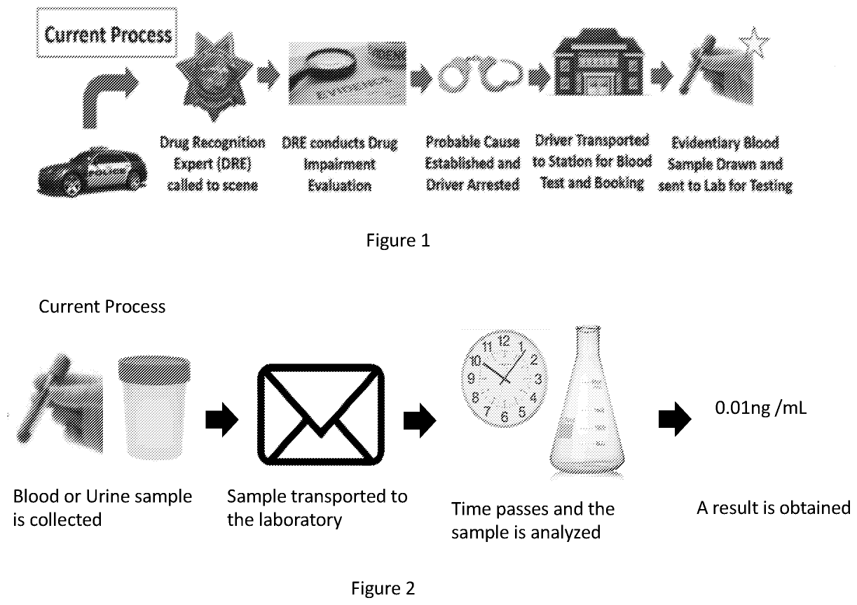

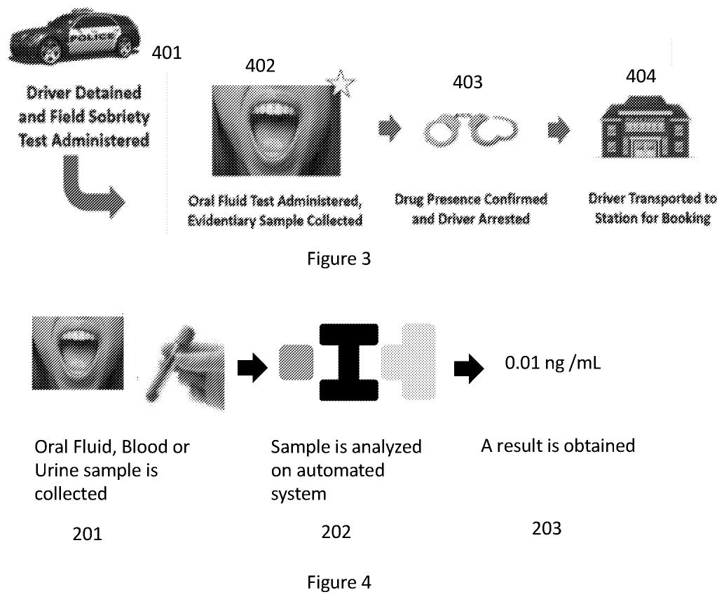

Referring to FIG. 3, a method as described herein for testing for impairing substances (e.g., during a roadside traffic stop) can include, at step 401, detaining the driver. At step 402, an oral fluid test can be administered, including gathering an evidentiary sample. At step 403, the presence of the impairing substance can be confirmed and, if necessary, the driver arrested. At step 404, the driver can be transported to the station for booking. Such a method can greatly reduce the number of steps required to evaluation impairment relative to the method shown in FIG. 1.

FIG. 4 shows an exemplary method as described herein of testing for impairing substances at workplace, medical establishments, or other settings. The method can include, at step 201, collecting an oral fluid, blood or urine sample, including an evidentiary sample. At step 202, the sample is automatically analyzed. At step 203, the presence of a substance can be confirmed and/or quantified. Such a method can greatly simplify the evaluation of impairment relative to the method shown in FIG. 2.

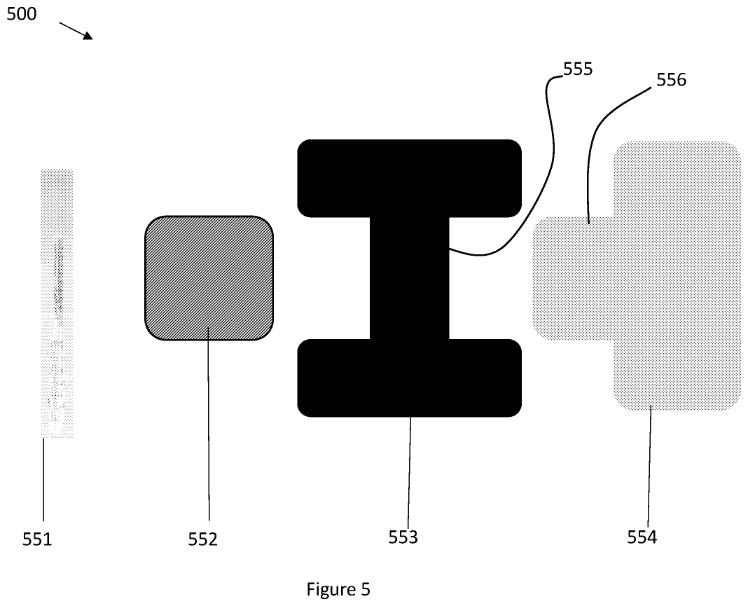

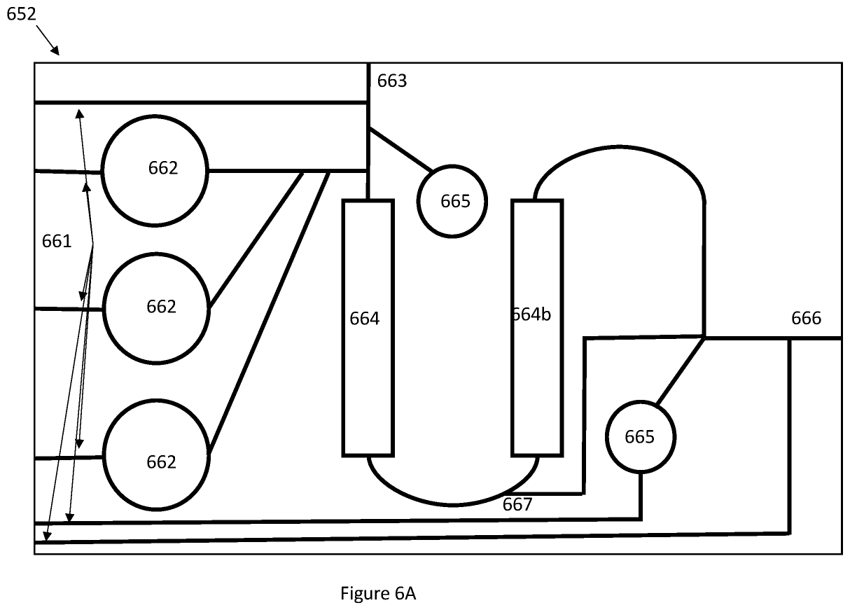

An exemplary system 500 for testing for impairing substances is shown in FIGS. 5. The system 500 includes a collector 551, a processing cartridge 552, a pumping system 553, and a detector 554.

The collector 551 can be configured to collect oral fluid (i.e., saliva), blood or urine. Exemplary collectors are described in U.S. Provisional Application No.: 62/718,851 and international patent publication WO 2020/036891, the entirety of which are incorporated by reference herein. Further, the collector 551 can be configured to mate with the cartridge 552 to transfer the collected fluid thereto. In some embodiments, the collector 551 can be fitted with a seal at the distal end thereof that can be pierced when connected to the cartridge 552.

The cartridge 552, in turn, can be configured to process the collected fluid (e.g., extract the compound for which testing is being performed from the collected fluid). The cartridge 552 can include, for example, layered beads therein configured to filter/extract the compound of interest (e.g., THC) from the collected fluid. Further, the cartridge 552 can be disposable or reusable. When reusable, the cartridge 552 can include automatic cleaning features therein configured to clean the chambers and elements therein between uses.

The cartridge 552 can mate with the pumping system 553. In some embodiments, the cartridge 552 and pumping system 553 can have registration guides to assist in mating the two together. In some embodiments, the registration guides can also identify the type of cartridge being used (e.g., which compound the cartridge is specifically designed to extract) such that the pumping system 553 and/or detector can automatically operate accordingly. The pumping system 553 can include one or more pumps to control gas and liquid flow through the cartridge 552. In some embodiments, the pumping system 553 can pump solvents/ reagents into the cartridge 552 (e.g., from external storage containers). In some embodiments, the pumping system 553 can pump the sample from one location in the cartridge 552 to another location in the cartridge 552 to process and prepare the sample for analysis. The pumping system 553 can include a syringe pump, a peristaltic pump, or other mechanical pump. In some embodiments, the pumping system 553 can include vacuum and pressurized gases to move liquids through the cartridge. In some embodiments, the pumping system 553 can include a series of valves to regulate the flow of fluid through the cartridge 552.

The pumping system 553 can mate with the detector 554 to transfer the extracted sample thereto for analysis. For example, an extension 556 on the detector 554 (and/or the entire detector 554) can fit within a slot 555 in the pumping system 553. The detector 554 can determine how much of the compound is present (e.g., provide a numerical output related to the level of THC in the saliva). In some embodiments, the pumping system 553 can be permanently attached to the detector 554. When permanently attached, the fluidic processor 553 and detector 554 can have additional cleaning options for reuse between samples.

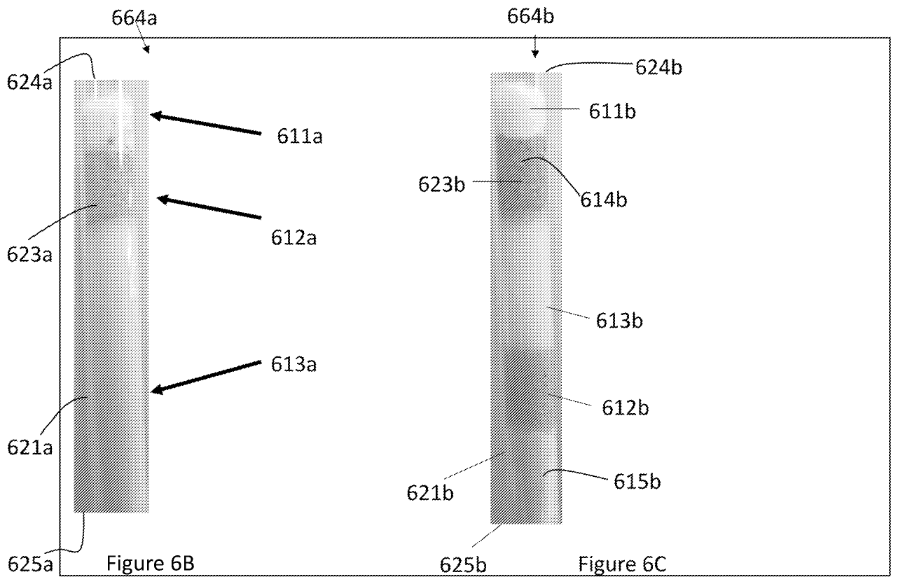

FIG. 6A is a diagram of an exemplary cartridge 652. The cartridge 652 advantageously allows one or more compounds (e.g., from the sample obtained by the collector) to be automatically and selectively isolated efficiently, precisely, and reliably. Samples processed by the cartridge 652 can be aqueous, organic, solids or gases. The cartridge 652 can include transfer lines 661 that can transfer gas, solid, and liquid samples, one or more reservoirs 662 for sample processing, a sample inlet 663 that can be mated with a sample collector, first and second phase transfer interface assemblies 664a and 664b that can filter/extract the compound of interest from the fluid, one or more sample archiving reservoirs 665 (e.g., for holding secondary samples for evidentiary purposes), and an outlet 666 to the pumping system and/or detector. In some embodiments, the cartridge 652 can include a valve 667 to allow for the bypassing the second phase transfer interface 664b. Further, in some embodiments, the sample inlet 663, transfer lines 661, and/or outlet 666 can have locking mechanisms (e.g., slip fittings) to ensure a leak free connection to the sample injection port, collector, or other sample holders. The sample reservoirs 662 and sample archiving reservoirs 665 may have a volume of 0.1-10 mL. The cartridge 652 may be constructed out of plastic or other durable material. The cartridge 652 may be disposable or reusable.

A. Phase Transfer Assembly

A close-up of exemplary phase transition interface assemblies 664a and 664b are shown in FIGS. 6B-6C. Each of the assemblies 664a, 664b can include a housing having multiple layers of beads that are configured to extract the compound of interest (e.g., THC).

For example, the first phase transfer interface assembly 664a of FIG. 6B includes a housing 621a having layered beads 623a therein. Each layer 611a, 612a, 613a can have a different functionality for extracting the compound of interest (e.g., THC). For example, layer 611a can be a filter that filters large particulate matter, layer 612a can be a selective binding layer, and layer 613a can be a phase transfer layer that acts to reversibly bind specific compounds. In some embodiments, isolation of one or more compounds of interest can occur by first loading the sample into the phase transfer interface 664b and then flowing a solvent through the phase transfer interface 664b from the proximal end 624a to the distal end 625a. The fluid can thus move through the filter 611a, then through the beads of layer 612a, and finally through the phase transfer interface beads at layer 613a (e.g., via force from the pumping system). The resulting extracted compound that exits at the distal end 625a can then be sent to the detector for analysis.

The phase transfer assembly typically comprises at least a filter layer and a phase transfer layer.

a) Filter Layer

The filter layer 611a can be used for samples that are heterogeneous mixtures, such as biological fluids, colloids, emulsions, or powdered/porous solids to prevent clogging of the phase transfer interface 664a. In some embodiments, the filter layer 611a can include beads that are made of one or more inert materials, such as polystyrene, sand or quartz sand. In other embodiments, the beads in filter layer 611a can be composed of chemically active materials designed to pretreat the samples for the rest of the cartridge/analysis (e.g., beads with C-18 coating, chiral coatings, derivatizing or chemical modification agents, or biological active compounds such as immune assays). The overall pore size of the filter layer 611a can be controlled by the size of the beads used, which can be spherical, shaped, or irregular, as long as they exhibit a narrow dispersion of sizes. Beads of the filter layer 611a can be 40-600 microns in diameter with a disparity of 20% or less. In some embodiments, the beads of filter layer 611a can be 40-60 microns in diameter. In other embodiments, the particles can be 300-600 microns. The filter layer 611a can advantageously allow for a single phase transfer interface 664a to accommodate any biological sample without prior preparation (e.g., because the filter can capture large particles in the open pores, capture interferants by sticking them to the large surface area, and/or have internal pores that collect compounds).

The pore size and subsequent choice of filter layers depends on the sample to be extracted. For a homogenous sample, no filter layer is required, however for heterogenous (i.e. colloid, or emulsion) samples one or more layers can be employed. For example, many biological samples contain both colloidal and emulsified components with widely varying sizes. Thus employing a single filter would either provide poor filtration or become clogged. The choice of the material depends on the desired extracting solvent analyte, as some filters could be chemically incompatible with either, or bind the analyte.

The filter layer can comprise media that filters out particles in sample having a size greater than any of 0.001 micron, 0.01 micron, 0.1 micron, 1 micron, 10 microns, or 100 microns.

b) Selective Binding Layer

In some embodiments, the selective binding layer 612a can include beads that bind chemicals or proteins that may interfere with analysis and/or prevent purification. For example, where gas chromatography is used as part of the detector, water can damage the column and interfere with analysis. In such a case, the beads of the binding layer 612a can be formed of desiccant materials, such as silica or molecular sieves, to capture the water (and therefore remove it from the extracted compound). As another example, where the collected fluid comprises biological material, biological matrices can prevent low limits of detection for a variety of analyses. In such a case, beads with functional groups such as such as, thiols, COOH, NH₃ or CHO can be used for the binding layer 612a to remove the biological matrix from the fluid (and thus from the final extracted compound). In other examples, beads of layer 612a can be functionalized to bind specific chemical groups or destroy compounds. In some embodiments, the selective binding layer 612a can be combined with the phase transfer layer 613a.

c) Phase Transfer Layer

Finally, in some embodiments, the phase transfer layer 613a can include media, e.g., beads, that reversibly bind compounds through physical or chemical interactions with different affinities depending on the surface and type of compound. Examples of phase transfer interface beads include quartz, alumina, polystyrene, silica, and beads functionalized with moieties such as C18, NH₃, or COOH. The beads in the phase transfer interface layer 613a can either retain or release compounds based on the affinity of the compound for the phase transfer interface material and solvent. A compound's affinity largely depends on the polarity of the solvent, thus by changing the solvent from polar (methanol) to non-polar (hexanes) allows for multiple different sets of compounds to be obtained or isolated (and this can be done several times). For example, flowing hexanes over a sample can release non-polar compounds (e.g., THC) from the phase transfer interface while the polar compounds are left behind. Alternately or additionally, the polar compounds (e.g., THCA) can collected by flowing a polar solvent, such as methanol through the phase transfer interface. Furthermore, the compound's affinity for both solvent and phase transfer interface material can be changed by protonate or deprotonate the compound, which can be controlled through the solvent's pH or inherent acid base nature of the solvent, for example triethyl amine. This allows the compound of interest to be collected while removing impurities. Furthermore, the amount of solvent required to remove a compound also varies with a compound's affinity for the phase transfer interface material of the beads. Compounds of interest can be further targeted by changing the affinity of a compounds for the phase transfer interface bead's surface. Furthermore, phase transfer interface beads can be utilized to control the PH and act as a buffer, which can also protonate or deprotonate a compound, thereby affecting a compound's affinity for solvent and beads. In some embodiments, the phase transfer interface beads of layer 613a can provide a surface area to volume ratio of at least 500,000 or greater, which provides for a minimum of approximately 1% of the total molecules to be situated at the surface (solvent interface) for immiscible solvents. The large number of surface molecules can advantageously allow for the rapid isolation of compounds with solvents that are miscible, slightly miscible, or completely immiscible.

As another example, the phase transfer interface assembly 664b of FIG. 6C includes a housing 621b having layered beads 623b therein. Each layer 611b, 614b, 613b, 612b, 615b can have a different functionality for extracting the compound of interest (e.g., THC). For example, layer 611b can be a filter layer that filters large particulate matter (similar to layer 611a) while layer 614b can be a second filter that is similar to layer 611b, but that has smaller beads. As a result, when fluid is filtered from the proximal end 624b to the distal end 625b, the fluid will encounter subsequently smaller beads, giving rise to pores that decrease in size from one layer to the next (which can advantageously prevent clogging). In the case of biological fluid, this may provide the ability to remove both cells and proteins from sample without clogging the filter. The layer 613b can be a phase transfer interface layer (similar to layer 613a), layer 612b can be a layer of binding beads (similar to layer 612a), and layer 615b can be a second layer of phase transfer interface beads (e.g., configured to bind different compounds than layer 613b).

In some embodiments, the materials used for the beads of the filter layers, binding layers, and phase transfer interface layers described herein can be composed of a diatomaceous earth, silica, quartz, glass, alumina, polystyrene, a variety of sands, such as sea sand or loamy sand, or other pulverized materials, such as a powdered metal (metal powder), ceramics, wood of cellulose. In some embodiments, these materials can also be functionalized in order to perform chemistry (carbodiimides), buffer a solution (control pH (ex. triethylamine)), or prepare a compound for a subsequent layer in the phase transfer interface 664 (cyanoborohydride, this can protonate or destroy unwanted things/change a compound to something known). Therefore, the phase transfer interfaces 664a,b, in addition to purification/isolation of compounds, can also be simultaneously used to perform synthetic chemistry. Functionalization of these materials can be performed through well known chemistry, which varies for each material.

In certain embodiments a selective binding layer that absorbs water, for example, a desiccant material, also functions as a phase transfer layer. As an organic solvent passes over particles having high surface area which adsorb water, and analyte soluble in the organic solvent transfers from the aqueous phase to the organic phase, where it can be extracted from the phase transfer assembly.

d) Extractions

Solvents used to extract analytes from the phase transfer assembly generally will be non-aqueous solvents, e.g., organic solvents.

For compounds that are neutral in the original sample to be extracted, such as THC from a biological system, a typical cartridge can be composed of a wire mesh, followed by quartz sand (filter), silica gel, and finally a wire mesh. For a neutral compound, a typical extraction can be performed by using a mid-polarity solvent, such as dichloromethane. Through this method selective isolation of one or more analytes, such as THC, can be achieved.

For analyte(s) that have the ability to carry one or more charges, enhanced extractions can be performed by controlling the charge of the analyte, which typically can be performed via adjusting the pH. The pH can be controlled either by adding a water soluble acid or base to the collector, or by mixing an organic acid, such as, lactic acid or benzoic acid, or an organic base, such as amines, phosphazenes, or guanidine to the extracting solvent.

A third method to control the pH can be achieved through the use of functionalized PTI beads. Examples of PTI beads with acidic functional groups include carboxylic acid functionalized, and p-Toluenesulfonic acid. Examples of PTI beads with basic functional groups include amines, phosphazenes, or guanidine. Finally, pre-treatment of normal PTI beads can also provide pH control, for example acidic or basic alumina. Accordingly, such materials can be included as a selective binding layer in the phase transfer assembly.

When using the typical PTI and extracting solvent described above, most efficient extractions occur when the analyte is neutral. For example most naturally occurring and synthetic psychoactive drugs are alkaloids (amphetamines, opioids, tryptamines, crack/cocaine) and at a biological pH often carry a charge and are retained on silica gel. By increasing the pH these compounds become neutral and can be readily extracted from silica gel.

In the event that one or more samples are desired or interferants/impurities become an issue, multiple extractions or washing steps can be performed on the cartridge by controlling the pH through the extracting solvent. Since each compound often exhibits a different pKₐ it is possible to selectively retain or isolate one more compounds through control of the pH. This can occur either by using several pH steps (i.e. changing the reservoir from which the solvent is pulled) or through a solvent gradient.

For charged analytes the PTI beads can be functionalized with non-polar moieties such as octadecane, or benzene and eluted using a mid-polarity solvent.

### II. Methods

By utilizing specific combinations and order of filter layers, binding layers, and phase transfer interface layers, the phase transfer interface assemblies described herein advantageously allow for the isolation of an enormous number of compounds. In some embodiments, a plurality of different phase transfer interface assemblies can be used to extract out a plurality of different compounds (and/or additional cartridges can be swapped out to provide analysis for additional compounds). In one exemplary embodiment, by loading an aqueous or biological sample (e.g., saliva) onto a cartridge including two phase transfer interfaces (e.g., both 664a and 664b), it is possible to perform a drug analysis, where compounds such as THC and oxycodone are acquired are separately, while THC-A is retained until more polar solvents such as MEOH are used as a second solvent. Advantageously, one cartridge can provide a minimum of at least three sets of compounds by changing the solvent and bead properties. In one exemplary embodiment, very polar solvent (e.g., methanol) is first added to the phase transfer interface first isolating the polar compounds. Second, a mid-polarity solvent (e.g., dichloromethane) is added to isolate the mid polarity analytes and finally a non-polar solvent (e.g., hexane) is added to the phase transfer interface to isolate the non-polar analytes.

Additionally, the phase transfer interface assemblies described herein can allow compounds to be transferred to a solvent of choice (e.g., miscible or immiscible), with or without any sample prep, using miscible, slightly miscible, or completely immiscible solvents. This transfer can be accomplished by loading the sample onto the phase transfer interface and then adding the solvent. In this process, the original solvent and interferents can be left behind and the analyte of interest can be transferred to the second solvent. Additionally, in some embodiments, multiple solvent reservoirs allow for reagents and solvents with different chemical properties to be used after the initial processing. For example, a very non-polar solvent and then a polar solvent can be used to transfer analytes with various properties.

Additionally, in some embodiments, the cartridge 652 can be configured to separate the extracted sample into separate samples—one for analysis and one for storage (e.g., for evidentiary purposes and/or further analysis). The second stored sample can be configured to be stored within the processor 600 until it is needed. The two samples can each be, for example, between 1 μL-10 mL in size.

An exemplary methods of this disclosure a sample comprising one or a plurality of analytes is loaded onto a phase transfer assembly that comprises a filter layer and a phase transfer layer. The filter layer can comprise a material that filters out particles in the sample having a size greater than, for example, 10 μm. Downstream of the filter layer can be the phase transfer layer comprising a desiccant, for example silica powder. As the aqueous phase of the sample passes into the silica powder water is absorbed to the silica particles. Then, an organic solvent in which the analyte is soluble passes through the phase transfer assembly, typically in the same direction as the sample is loaded. As the organic solvent passes into the phase transfer layer analyte molecules are transferred into the organic solvent. Continual flow of the organic solvent into the phase transfer assembly extracts the analyte from the phase transfer assembly.

In another exemplary method a sample comprises a first and a second analyte. The phase transfer assembly comprises a filter layer, a selective binding layer and a phase transfer layer. In one embodiment the selective binding layer comprises a silica powder to function as a desiccant and the phase transfer layer comprises a selective binding material that binds the first analyte but not the second analyte when dissolved in a first but not a second organic solvent. After the sample is loaded and passed through the selective binding layer, a first organic solvent elutes the two analytes into the phase transfer layer. There, the first analyte is bound and the second analyte is eluted from the phase transfer assembly. Then, a second organic solvent is passed through the phase transfer assembly. The second organic solvent is chosen so that the second analyte is extracted from the phase transfer layer and is further extracted from the phase transfer assembly.

### III. System

Referring back to FIG. 5, the cartridge 552 (or 652) can be configured to be placed within a portable detector 554) The detector 554 can obtain the extracted sample through a sample handling line fluidically connecting the cartridge 552/652 to the detector 554 and can be configured to analyze for the presence of chemicals. The detector 554 can use, for example, a separation technique including column chromatography, ion-exchange chromatography, gel-permeation (molecular sieve) chromatography, affinity chromatography, gas chromatography, paper chromatography, thin-layer chromatography, dye-ligand chromatography, hydrophobic interaction chromatography, pseudoaffinity chromatography, high-pressure/performance liquid chromatography (HPLC), ion mobility, vacuum chromatography, or flash chromatography. Further, the detector can use a detection technique including an ion mobility detector (IM), a charged aerosol detector (CAD), a flame ionization detector (FID), an aerosol-based detector (NQA), a flame photometric detector (FPD), an atomic-emission detector (AED), a nitrogen phosphorus detector (NPD), a photo ionization detector (PID), an evaporative light scattering detector (ELSD), a mass spectrometer (MS), an electrolytic conductivity detector (ELCD), a sumon detector (SMSD), a mira detector (MD), an ultraviolet (UV) detector, a thermal conductivity detector, a fluorescence detector, an electron capture detector (ECD), a conductivity monitor, a refractive index detector (RI or RID), a radio flow detector, a chiral detector, a microelectromechanical (MEMS) detector, a cantilever detector, a photomultiplier tube (PMT) detector, a mass selective detector, a micro channel plate detector, or an electron multiplier detector. The results of the detection mechanism can be quantitative and can be accurate at concentrations of 0.001 ng/ml and greater, such as 0.001 ng/ml and greater. The detection limit can thus be below 0.01 ng/ml. In some embodiments, the detector can include a concentrator device that will allow the analyte to be concentrated to a small volume. For example, the sample may be heated, or the pressure lowered to remove any solvent and concentrate the analyte. Other techniques of sample concentration include solid phase micro extraction (SPME), solid phase extraction (SPE), and sorption.

In some embodiments, the results from the analysis can be displayed in a quantitative display on the detector or device. Such quantitation can be obtained through methods such as internal standards, calibration data, and total ion signal. In some embodiments, the sample can be mass selected, and an internal standard of the analyte can be isotopically labeled. Additionally, in some embodiments, the results can be sent to a phone and/or remote computer or cloud for further analysis or storage.

In some embodiments, the cartridge can be configured to add additional reagents for derivatization and other chemical modification of the sample.

The detector (and the attached processor) can be configured to fit within a case, e.g., for placement in the trunk of a police car. The overall device, therefore can be less than 3,000 cubic inches, such as less than 30 L in volume. In some embodiments, the detector can use less than 300 watts of power. Further, the detector (and entire device) can be lightweight, e.g., weigh less than 10 kg.

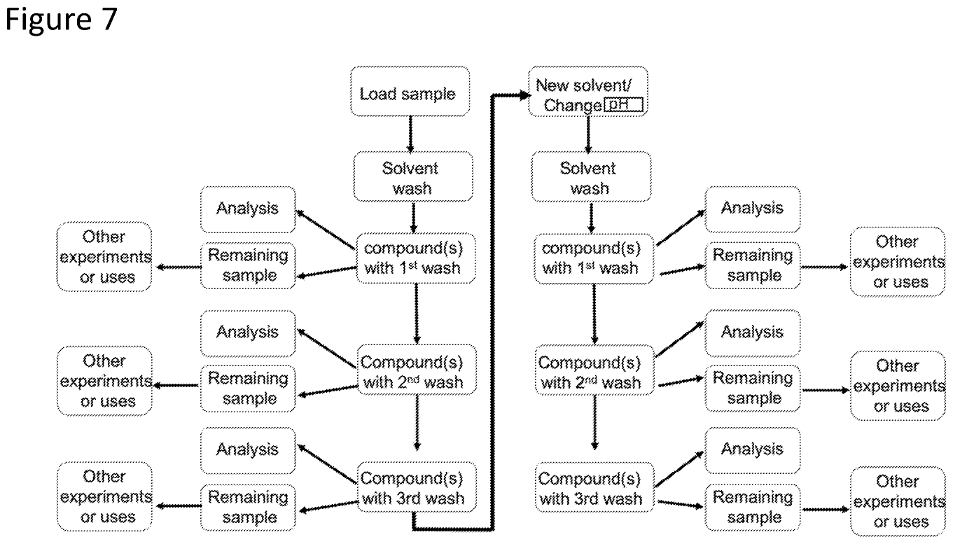

An exemplary overall method of gathering sample through analysis is shown in FIG. 7. In some embodiments, the steps that occur after collection through analysis can take 10 minutes or less. The devices, systems, and methods described herein can advantageously result in a quantitative test for impairing substances (e.g., THC) directly at the location (e.g., roadside) in less than 15 minutes from the start of the analysis (e.g., the user chewing on the bulb). Additionally, a sample can be safely and efficiently stored for later evidentiary use.

The method of concentrating the sample and the phase transfer process through a phase transfer interface as described herein can take less than 5 minutes. The total analysis time can be under 15 minutes.

In addition to drug testing, the systems and methods described herein can be used to prepare any aqueous sample by concentrating and transferring the solutes into an organic solvent. For example, the systems described herein can be used to test a sample for pesticides, hormones, and small molecules. The device can also prepare samples that are in an organic solvent and samples that are solids or gases. This application for research and chemical monitoring is a rapid and efficient method for sample preparation. For example, a water sample containing pesticides can be prepared for analysis in an organic phase using the device described herein.

### EXAMPLES

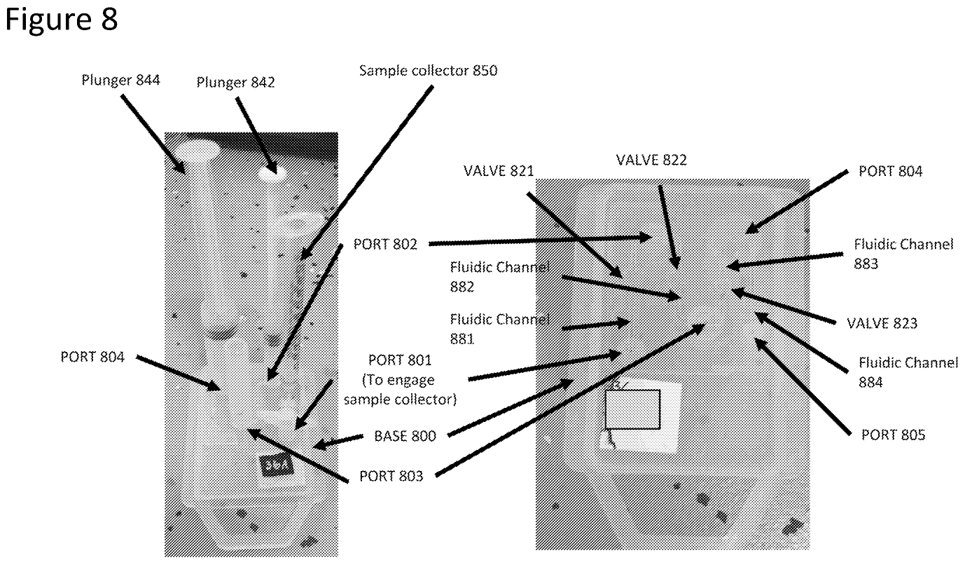

FIGS. 8-10 depict an exemplary cartridge of this disclosure. The cartridge 100 comprises a base 800 and a cover 900. FIG. 8 depicts a top-down view of base 800 including various chambers and plungers. Accordingly, certain chambers are compartments of the cartridge can be configured as syringes comprising a barrel in a plunger inside the barrel. It also depicts a bottom-face view of base 800, showing various fluidic elements. The ports open on the top, rather than the bottom face of base 800, but they are visible through the transparent bottom face of the plastic base.

The base 800 comprises port 801 configured to engage an exit port of sample collector 850. The engagement can be via a friction fit, a screw-in device, a taper fit, luer lock, snap, etc. An exemplary sample collector is described in international patent publication WO 2020/036891, published Feb. 20, 2020 entitled “Portable drug testing apparatus, system, and method”, which is incorporated herein by reference in its entirety.

Port 801 communicates through a fluidic channel 881 in the base with port 802. Liquid flow through channel 881 may be controlled by valve 821.

Valves used in exemplary cartridges can be any type of valve typically used in microfluidic devices. Valves can be actuated, for example, mechanically, pneumatically, or electrokinetically. For example, the valve can be a diaphragm or pinch valve which closes when one surface of the valve is pressed against another surface to occlude a channel. In other embodiments, the valve can be a rotary valve which, in one orientation blocks flow and which, in another and orientation connects passages to allow flow. In another embodiment the valve is a phase change valve formed for example of paraffin in which melting paraffin open channel to allow liquid flow.

Port 802 communicates on a top face of base 800 with sample chamber 862, adapted here to accept plunger 842. Port 802 further communicates with port 803 by a fluidic channel 882. Liquid flow through channel 882 may be controlled by valve 822.

Port 803 communicates on a top face of base 800 with phase transfer assembly 863. Phase transfer assembly 863 comprises an open top (port 893). Port 803 further communicates with port 804 by a fluidic channel 883. Liquid flow through channel 883 may be controlled by valve 823.

Port 804 communicates on the top face of base 800 with solvent chamber 864. Solvent chamber 864 comprises an open top 894 configured to accept plunger 844. Port 804 further communicates through the channel 884 with Port 805, adapted to engage a port in the instrument upon mating.

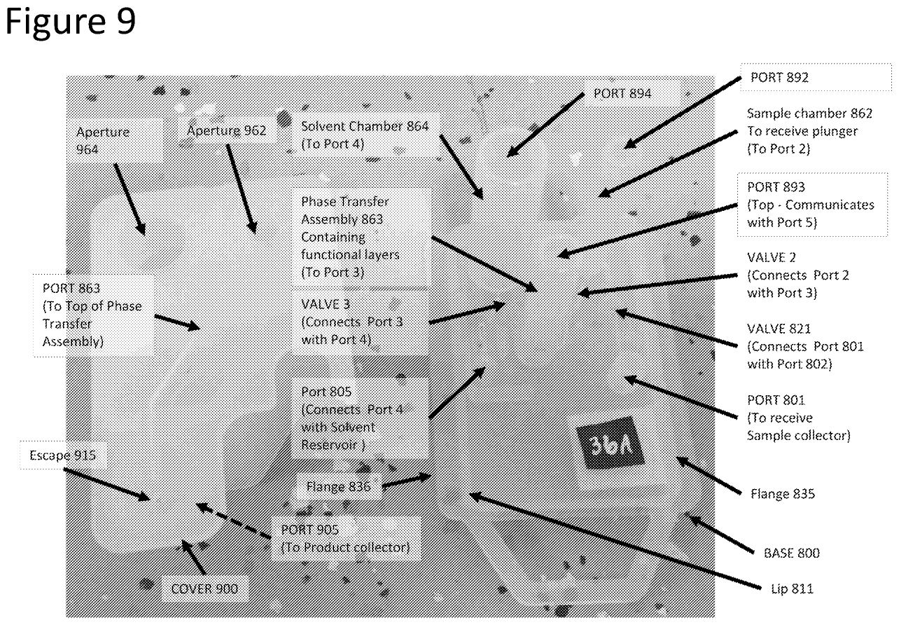

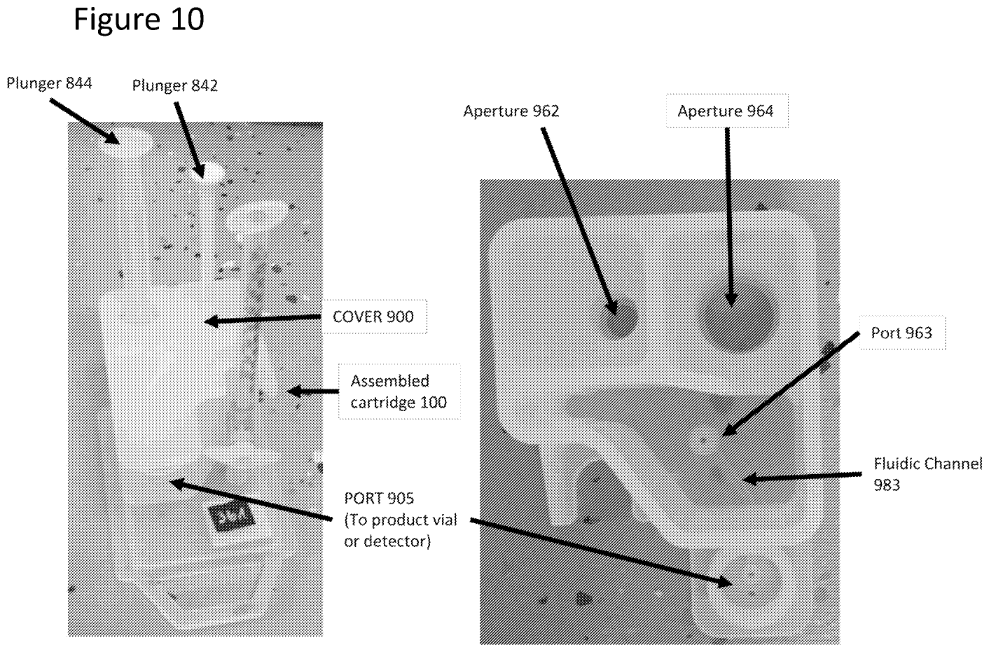

Referring to FIGS. 9 and 10, the cartridge further comprises cover 900, shown from a top face view in FIGS. 9 and 10, and from the bottom face view in FIG. 10. FIG. 10 further shows cover 900 engaged with base 800, as an assembled unit. Cover 900 comprises aperture 964 configured to fit as a sleeve over solvent chamber 864 and aperture 962 configured to fit as a sleeve over sample chamber 862. On a bottom face of cover 900, port 963 communicates with phase transfer chamber 862 when cover 900 is engaged with base 800. Port 963 further communicates through fluidic channel 993 with port 905 on the underside of the cover, through which analyte can be eluted from the cartridge. Cover 900 also can comprise an escape port 915 from which gas can escape. A lip or rim 811 on the base can serve as a friction fit or snap to secure cover 900 to base 800.

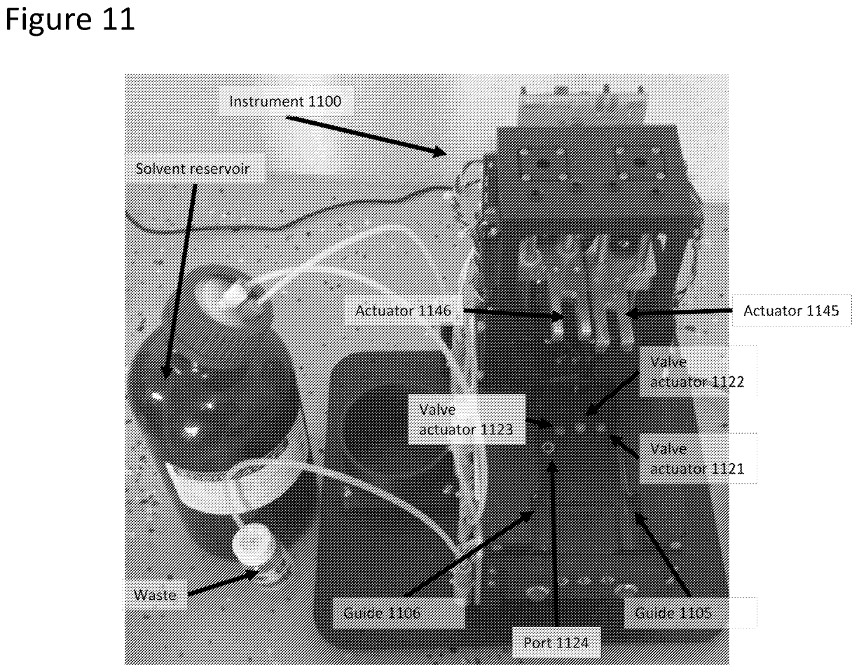

Referring to FIG. 11, an exemplary system of this disclosure comprises instrument 1100 which is configured to engage flanges 835 and 836 of cartridge 100 through guides 1105 and 1106. Valve actuators 1121, 1122, and 1123 align with valves 801, 802, and 803, respectively. The actuators can be selected to actuate the type of valve in the cartridge. For example, if the cartridge valve is a pinch valve or diaphragm valve, the actuator can be a piston or rod which presses against the valve. Such pistons or rods can be moved by, for example, solenoids or stepper motors. Port 1124 in the instrument is positioned to mate with port 804 in the cartridge. This forms a line of fluid communication between solvent reservoir and a solvent chamber in the cartridge. This line can also comprise a spur separated by a valve to a waste chamber, for example, for priming the solvent pump. Pump actuators 1145 and 1146 push down or pull up plungers 842 and 844, respectively and are moved by motors in the instrument control, for example, electrically.

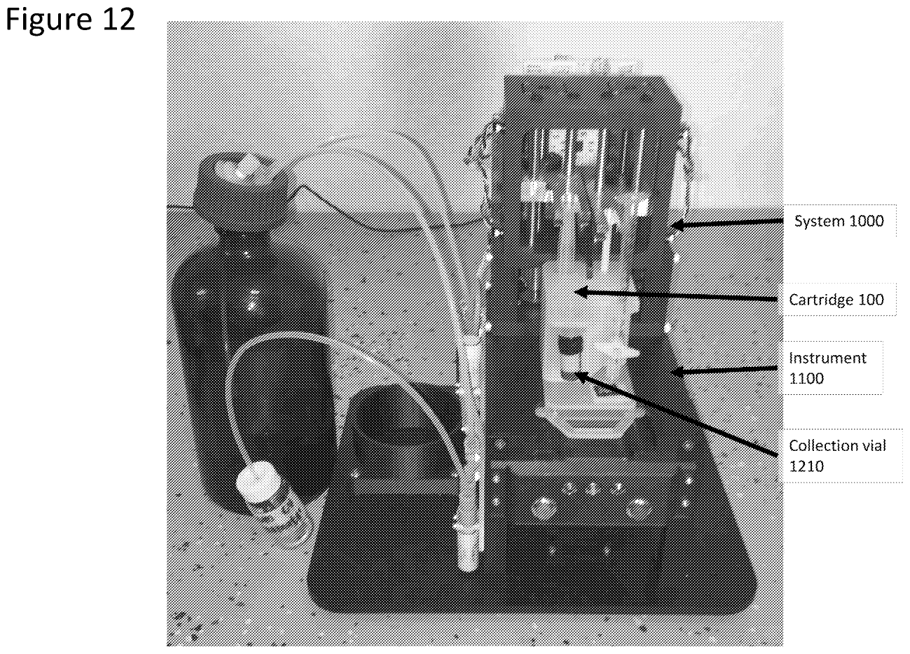

Referring to FIG. 12, system 1000 comprises instrument 1100 and cartridge engaged with the instrument. Analyte eluted from the cartridge can be collected in a collection vial 1210.

System 1100 can further comprise a mechanical assembly comprising a motor to move certain actuators, and solenoids to move other actuators. An electrical assembly can be connected to a power supply and to electrically powered components of the instrument.

System 1100 can further comprise a programmable digital computer. Computer systems can include a processor and memory accessible by the processor. For example, the processor can be a central processing unit (CPU). Memory can be in tangible form, for example, read only memory or random-access memory. Memory can include machine executable code (e.g., software) that, when executed by the processor, carries out instructions in the code. The software can, for example, include instructions for executing methods of this disclosure by operations of the system.

The computer system can also include a communication interface (e.g., network adapter) for communicating with one or more other systems, and peripheral devices. The computer system can be in communication with a computer network, such as a local area network or the internet. The system can be in communication with the internet through, for example, a high-speed transmission network including, without limitation, Digital Subscriber Line (DSL), Cable Modem, Fiber, Wireless, Satellite and, Broadband over Powerlines (BPL). The computer system also can comprise a display or a user interface, such as a graphic user interface, for interacting with the computer.

In other embodiments, the cartridge does not comprise one or more chambers, for example, the sample chamber or a solvent chamber. Rather, these chambers are included in the cartridge interface of the instrument, where they communicate directly with corresponding cartridge ports (e.g., Port 802 or port 804). The cartridge can include a solvent reservoir. The instrument can include syringes or plungers that engage the solvent chamber or the sample chamber on the cartridge. The system also can comprise a detector which communicates directly with an exit port, e.g., port 905 of the cartridge.

An exemplary operation of the system proceed as follows:

1. A new cartridge is placed into the system and all valves are closed

2. Sample is collected using a sample collector device

3. The sample collector device is then engaged with port 801

4. The “go” button is then pressed and valve 821 opens and a known amount of sample is pulled (measured) from the sample collector device into the sample chamber 862 by pulling up on the plunger 842

5. For solid samples a pre-extraction set can be employed, e.g., put solvent into sample collector

6. Valve 821 is closed and Valve 822 is opened, and a known amount of sample is dispensed from port 802 into port 803 and into phase transfer assembly 863 by pushing down on the plunger 842

7. Valve 822 is closed and valve 824 is opened and a known amount of solvent is drawn into solvent chamber 864 through port 804 by pulling up on the plunger 844.

8. Valve 824 is closed and valve 823 is opened and a known amount of solvent stored in solvent chamber 864 is dispensed from port 804 by pushing down on the plunger 844. The solvent is pushed through port 803 into phase transfer assembly 863.

9. The solvent then elutes from phase transfer assembly 863 through port 963 into channel 983 and out port 905, where it can then either be directly connected to a detector or be collected for storage

10. In the event that an extraction requires multiple steps, then the solvent reservoir can be changed and steps 6-9 are repeated until the desired results are achieved.

### EXEMPLARY EMBODIMENTS

1. A system comprising: I) an instrument comprising a cartridge interface configured to engage a cartridge of embodiment 5 or embodiment 33 or as disclosed herein; and II) a cartridge of embodiment 5 or embodiment 33 as disclosed herein engaged with the cartridge interface. 2. The system of embodiment 1, wherein: A) the cartridge interface comprises one or more of: 1) one or more valve actuators positioned to actuate valves in the cartridge; 2) a port positioned to communicate with the solvent transfer interface port; 4) a guide to accept an alignment mechanism in the cartridge; 5) one or more pump actuators positioned to actuate one or more pumps in the cartridge; or one or more ports communicating with one or more compartments in the cartridge and with one or more pumps in the instrument to impart positive or negative pressure to the compartments; 6) a container positioned to receive liquid eluted from an exit port in the cartridge; and 7) a port configured to engage an exit port in the cartridge and communicating through a fluidic channel with a detector. 3. The system of embodiment 1 or embodiment 2, wherein: B) the instrument further comprises one or more of: 1) a solvent reservoir comprising an organic solvent, wherein the solvent reservoir communicates through a fluidic channel with the solvent chamber of the cartridge; 2) one or more pumps to pump the organic solvent from the solvent reservoir to the solvent chamber; 3) one or more sources of positive and/or negative pressure configured to communicate pressure to the sample chamber and or the solvent chamber of the cartridge; 4) one or more motors to actuate one or more pumps in the cartridge (e.g., apply positive or negative pressure to a plunger); 5) one or more pumps, which pumps provide positive and/or negative pressure to one or more compartments in the cartridge; 6) an electrical assembly connectable to a source of electric power, which electrical assembly provides power to one or more motors and the one or more sources of positive or negative pressure; 7) a computer comprising software and a processor which executes software to control operation of the elliptical assembly, the one or more motors and the one or more sources of positive or negative pressure; and 8) a detector communicating with an exit port on the cartridge to detect analyte eluted from the cartridge. 4. The system of embodiment 3, wherein the valve actuators comprise solenoids or stepper motors.

5. A cartridge for sample preparation comprising: an inlet configured to receive a collected sample; a phase transfer assembly comprising a plurality of bead layers, wherein the collected sample is configured to be transferred through the bead layers of the phase transfer assembly to extract a compound therefrom; and an outlet configured to transfer the extracted compound to a container or a detector for analysis of the extracted compound. 6. The cartridge of embodiment 5, wherein the extracted compound is THC. 7. The cartridge of embodiment 5, wherein the cartridge is disposable. 8. The cartridge of embodiment 5, further comprising one or more ports communicating the phase transfer assembly, and configured to engage a port of a cartridge interface of an instrument. 9. The cartridge of embodiment 5, further comprising one or more registration guides configured to mate with a pumping system or the detector. 10. The cartridge of embodiment 5, further comprising one or more reservoirs configured to store the collected sample. 11. The cartridge of embodiment 5, further comprising one or more reservoirs configured to store the extracted compound. 12. The cartridge of embodiment 5, further comprising a second phase transfer assembly comprising a plurality of bead layers configured to extract a second compound from the collected sample. 13. The cartridge of embodiment 5, further the plurality of bead layers comprises a first layer and a second layer, wherein the first layer comprises a filter layer, and the second layer comprises a selective binding layer. 14. The cartridge of embodiment 13, wherein the plurality of bead layers further comprises a third layer comprising a phase transfer layer. 15. The cartridge of embodiment 5, wherein the plurality of bead layers comprise a first layer, the first layer comprising a filter layer. 16. The cartridge of embodiment 15, wherein the filter layer comprises beads between 40 and 600 microns in diameter. 17. The cartridge of embodiment 15, wherein the filter layer comprises beads with 20% or less disparity in diameter. 18. The cartridge of embodiment 15, wherein the filter layer comprises inert beads. 19. The cartridge of embodiment 18, wherein the inert beads comprise polystyrene, sand, or quartz sand. 20. The cartridge of embodiment 15, wherein the filter layer comprises chemically active beads. 21. The cartridge of embodiment 20, wherein the chemically active beads comprise beads with a C-18 coating, a chiral coating, a derivatizing or chemical modification agent, or a biological active compound. 22. The cartridge of embodiment 5 or 15, wherein the plurality of bead layers comprise a second layer, the second layer comprising a selective binding layer. 23. The cartridge of embodiment 22, wherein the selective binding layer comprises beads comprising a desiccant. 24. The cartridge of embodiment 22, wherein the selective binding layer comprises beads comprising a thiol, COOH, NH3, or CHO. 25. The cartridge of embodiment 5 or 15 or embodiment 22, wherein the plurality of bead layers comprises a third layer, the third layer comprising a phase transfer layer. 26. The cartridge of embodiment 25, wherein the phase transfer layer comprises beads that are configured to reversibly bind compounds. 27. The cartridge of embodiment 26, wherein the beads of the phase transfer layer comprise quartz, alumina, polystyrene, or silica. 28. The cartridge of embodiment 21, wherein the beads of the phase transfer layer comprise beads functionalized with C-18, NH3, or COOH. 29. The cartridge of embodiment 25, wherein the beads of the phase transfer layer provide a surface area to volume ratio of at least 500,000 or greater. 30. The cartridge of embodiment 5, wherein the plurality of beads layers comprises a first filter layer and a second filter layer, the first filter layer having beads of higher diameter than beads of the second filter layer. 31. The cartridge of embodiment 5, wherein the phase transfer assembly comprises a filter layer comprising media configured to filter out particles having a size greater than 0.001 μm, 0.01 μm, 0.1 μm, 1 μm, 10 μm or 100 μm; and a selective binding layer comprising a desiccant. 32. The cartridge of embodiment 5, wherein the cartridge comprises a plurality of ports, each port communicating with a chamber when the cartridge is engaged with the cartridge interface of an instrument, and wherein each port further communicates with the phase transfer assembly.

33. A cartridge comprising: a) a sample collector port comprising a receptacle configured to engage a sample collector, e.g., through a fiction fit or a screw mechanism; b) a sample chamber, optionally comprising a liquid sample comprising an analyte, communicating with the sample collector port through a fluidic channel, wherein fluid flow between the sample chamber and the sample collector port is optionally regulated by a first valve; c) a phase transfer compartment comprising a bottom port and a top port, optionally comprising a layer of media for removal of particulates and a desiccant layer, communicating with the sample chamber port through a fluidic channel at the bottom port, wherein fluid flow between the phase transfer compartment and the sample chamber is optionally regulated by a second valve; c) a solvent chamber, optionally comprising an organic solvent, communicating with the phase transfer compartment through a fluidic channel at the bottom port, wherein fluid flow between the solvent chamber and the phase transfer compartment is optionally regulated by a third valve; e) a solvent transfer interface port communicating with the solvent chamber through a fluidic channel and configured to engage an instrument comprising a solvent source; and f) a product port communicating with the phase transfer assembly through a fluidic channel at the top port. 34. The cartridge of embodiment 33, comprising a base and a cover, wherein the base comprises the fluidic channels communicating between the sample collector port, sample chamber, the phase transfer compartment and the solvent chamber, and a cover comprises the fluidic channel communicating between the product port and the phase transfer chamber. 35. The cartridge of embodiment 34, wherein the cover comprises sleeves that fit over each of sample chamber, the phase transfer assembly and the solvent chamber. 36. The cartridge of embodiment 33, wherein the sample chamber and the solvent chamber are configured as pumps comprising a drive to impart positive and/or negative pressure to the chamber compartment. 37. The cartridge of embodiment 36, wherein the drive comprises a plunger. 38. The cartridge of embodiment 34, wherein the cover is secured on the base through a snap fit or a friction fit. 39. The cartridge of embodiment 33, comprising a handle. 40. The cartridge of embodiment 33, comprising a guide or key to engage a cartridge interface of an instrument.

41. A method comprising: a) providing a sample comprising a first analyte; b) loading the sample onto a first phase transfer assembly, wherein the first phase transfer assembly comprises: i) a first filter layer comprising media configured to filter out particulate matter having a size of at least 0.1 microns, 1 μm, 10 μm, or 100 μm; and ii) a selective binding layer comprising a desiccant configured to adsorb water from the sample; and c) eluting the first analyte from the first phase transfer assembly by flowing a non-aqueous solvent through the first phase transfer assembly. 42. The method of embodiment 41, wherein the first phase transfer assembly further comprises: iii) a first phase transfer layer comprising media that binds the analyte; and c) eluting comprises: i) flowing a first solvent through the first phase transfer assembly to elute unbound material in a first eluate; and ii) flowing a second solvent through the first phase transfer assembly to extract the first analyte bound to the media therein. 43. The method of embodiment 42, further comprising: d) loading the first eluate onto a second phase transfer assembly, where the second phase transfer assembly comprises: iii) a second phase transfer layer comprising media that binds a second analyte in the first eluate; e) eluting the second analyte from the second phase transfer assembly by flowing a solvent through the second phase transfer assembly to extract the second analyte bound to the media therein. 44. The method of embodiment 43, further comprising: d) loading the first eluted analyte onto a second phase transfer assembly, where in the second phase transfer assembly comprises: 45. The method of embodiment 43, wherein the sample is loaded at a bottom of the phase transfer assembly and eluted from a top of the phase transfer assembly. i) a second filter layer comprising particles configured to filter out particulate matter having a size of at least 0.01 microns from the sample; and ii) a second selective binding layer a desiccant configured to adsorb water from the sample; and iii) a second phase transfer layer comprising media that binds the second analyte; and e) eluting the second analyte from the phase transfer assembly with a first organic solvent and the second analyte from the first with a first. 46. The method of embodiment 42, wherein the phase transfer layer binds the analyte, and extracting the analyte comprises changing the pH of the phase transfer layer so that the analyte no longer binds. 47. The method of embodiment 43, wherein the sample comprises a first and a second analyte, wherein the media in the phase transfer layer binds the second analyte, and wherein eluting comprises eluting the first analyte with a first organic solvent and eluting the second analyte with a second organic solvent. 48. The method of embodiment 41, wherein the sample is an aqueous sample comprising a cannabinoid, e.g., tetrahydrocannabinol (“THC”), the first filter layer comprises silica sand (e.g., about 220 micron), the selective binding layer comprises silica gel (e.g., 40-60 microns) and non-aqueous solvent comprises dichloromethane. 49. The method of embodiment 41, wherein the sample is an aqueous sample comprising cocaine or an opioid, the first filter layer comprises silica sand (e.g., about 220 micron), the selective binding layer comprises silica gel (e.g., 40-60 microns) and non-aqueous solvent comprises a dichloromethane/methanol mixture or dichloromethane/tributylamine mixture. 50. The method of embodiment 41, further comprising detecting the analyte in the eluate.

51. A method comprising: a) moving a liquid sample comprising an analyte from a sample collector into a sample chamber; b) moving the liquid sample from the sample chamber into a phase transfer assembly; c) moving an organic solvent from a solvent chamber into the phase transfer assembly; and d) eluting the analyte from the phase transfer assembly with the organic solvent. 52. The method of embodiment 51, further comprising: e) moving an organic solvent from a solvent reservoir into the solvent chamber. 53. The method of embodiment 51, further comprising detecting the eluted analyte with a detector. 54. The method of embodiment 51, wherein moving a liquid sample comprising an analyte from a sample collector into a sample chamber comprises applying negative pressure. 55. The method of embodiment 51, wherein moving the liquid sample from the sample chamber into a phase transfer assembly comprises applying positive pressure. 56. The method of embodiment 51, wherein moving the organic solvent from the solvent chamber into the phase transfer assembly comprises applying positive pressure. 57. The method of embodiment 51, wherein eluting the analyte from the phase transfer assembly comprises applying positive pressure. 58. The method of embodiment 51, wherein moving the organic solvent from the solvent reservoir into the solvent chamber comprises applying negative pressure. 59. The method of embodiment 51, performed by a system of embodiment 1.

It should be understood that any feature described herein with respect to one embodiment can be substituted for, or used in addition to, any feature described herein with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In certain embodiments, an invention that comprises elements may consist essentially of these elements. The term “consisting essentially of” refers to the inclusion of recited elements and other elements that do not materially affect the basic and novel characteristics of a claimed combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

The term “any of” between a modifier and a sequence means that the modifier modifies each member of the sequence. So, for example, the phrase “at least any of 1, 2 or 3” means “at least 1, at least 2 or at least 3”.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.”

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

All publications, patents, and patent applications mentioned in this specification are herein incorporated by reference to the same extent as if each individual publication, patent, or patent application was specifically and individually indicated to be incorporated by reference.

What is claimed is:

1. A system comprising:
I) an instrument comprising a cartridge interface configured to engage a cartridge of claim 5 or claim 33; and
II) a cartridge of claim 5 or claim 33 engaged with the cartridge interface.

2. The system of claim 1, wherein:
A) the cartridge interface comprises one or more of:
1) one or more valve actuators positioned to actuate valves in the cartridge;
2) a port positioned to communicate with the solvent transfer interface port;
4) a guide to accept an alignment mechanism in the cartridge;
5) one or more pump actuators positioned to actuate one or more pumps in the cartridge; or one or more ports communicating with one or more compartments in the cartridge and with one or more pumps in the instrument to impart positive or negative pressure to the compartments
6) a container positioned to receive liquid eluted from an exit port in the cartridge; and
7) a port configured to engage an exit port in the cartridge and communicating through a fluidic channel with a detector.

3. The system of claim 1 or claim 2, wherein:
B) the instrument further comprises one or more of:
1) a solvent reservoir comprising an organic solvent, wherein the solvent reservoir communicates through a fluidic channel with the solvent chamber of the cartridge;
2) one or more pumps to pump the organic solvent from the solvent reservoir to the solvent chamber;
3) one or more sources of positive and/or negative pressure configured to communicate pressure to the sample chamber and or the solvent chamber of the cartridge;
4) one or more motors to actuate one or more pumps in the cartridge (e.g., apply positive or negative pressure to a plunger);
5) one or more pumps, which pumps provide positive and/or negative pressure to one or more compartments in the cartridge;
6) an electrical assembly connectable to a source of electric power, which electrical assembly provides power to one or more motors and the one or more sources of positive or negative pressure;
7) a computer comprising software and a processor which executes software to control operation of the elliptical assembly, the one or more motors and the one or more sources of positive or negative pressure;
8) a detector communicating with an exit port on the cartridge to detect analyte eluted from the cartridge.

4. The instrument of claim 3, wherein the valve actuators comprise solenoids or stepper motors.

5. A cartridge for sample preparation comprising:
an inlet configured to receive a collected sample;
a phase transfer assembly comprising a plurality of bead layers, wherein the collected sample is configured to be transferred through the bead layers of the phase transfer assembly to extract a compound therefrom; and
an outlet configured to transfer the extracted compound to a container or a detector for analysis of the extracted compound.

6. The cartridge of claim 5, wherein the extracted compound is THC.

7. The cartridge of claim 5, wherein the cartridge is disposable.

8. The cartridge of claim 5, further comprising one or more ports communicating the phase transfer assembly, and configured to engage a port of a cartridge interface of an instrument.

9. The cartridge of claim 5, further comprising one or more registration guides configured to mate with a pumping system or the detector.

10. The cartridge of claim 5, further comprising one or more reservoirs configured to store the collected sample.

11. The cartridge of claim 5, further comprising one or more reservoirs configured to store the extracted compound.

12. The cartridge of claim 5, further comprising a second phase transfer assembly comprising a plurality of bead layers configured to extract a second compound from the collected sample.

13. The cartridge of claim 5, further the plurality of bead layers comprises a first layer and a second layer, wherein the first layer comprises a filter layer, and the second layer comprises a selective binding layer.

14. The cartridge of claim 13, wherein the plurality of bead layers further comprises a third layer comprising a phase transfer layer.

15. The cartridge of claim 5, wherein the plurality of bead layers comprise a first layer, the first layer comprising a filter layer.

16. The cartridge of claim 15, wherein the filter layer comprises beads between 40 and 600 microns in diameter.

17. The cartridge of claim 15, wherein the filter layer comprises beads with 20% or less disparity in diameter.

18. The cartridge of claim 15, wherein the filter layer comprises inert beads.

19. The cartridge of claim 18, wherein the inert beads comprise polystyrene, sand, or quartz sand.

20. The cartridge of claim 15, wherein the filter layer comprises chemically active beads.

21. The cartridge of claim 20, wherein the chemically active beads comprise beads with a C-18 coating, a chiral coating, a derivatizing or chemical modification agent, or a biological active compound.

22. The cartridge of claim 5 or 15, wherein the plurality of bead layers comprise a second layer, the second layer comprising a selective binding layer.

23. The cartridge of claim 22, wherein the selective binding layer comprises beads comprising a desiccant.

24. The cartridge of claim 22, wherein the selective binding layer comprises beads comprising a thiol, COOH, NH3, or CHO.

25. The cartridge of claim 5 or 15 or claim 22, wherein the plurality of bead layers comprises a third layer, the third layer comprising a phase transfer layer.

26. The cartridge of claim 25, wherein the phase transfer layer comprises beads that are configured to reversibly bind compounds.

27. The cartridge of claim 26, wherein the beads of the phase transfer layer comprise quartz, alumina, polystyrene, or silica.

28. The cartridge of claim 21, wherein the beads of the phase transfer layer comprise beads functionalized with C-18, NH3, or COOH.

29. The cartridge of claim 25, wherein the beads of the phase transfer layer provide a surface area to volume ratio of at least 500,000 or greater.

30. The cartridge of claim 5, wherein the plurality of beads layers comprises a first filter layer and a second filter layer, the first filter layer having beads of higher diameter than beads of the second filter layer.

31. The cartridge of claim 5, wherein the phase transfer assembly comprises a filter layer comprising media configured to filter out particles having a size greater than 0.001 μm, 0.01 μm, 0.1 μm, 1 μm, 10 μm or 100 μm; and a selective binding layer comprising a desiccant.

32. The cartridge of claim 5, wherein the cartridge comprises a plurality of ports, each port communicating with a chamber when the cartridge is engaged with the cartridge interface of an instrument, and wherein each port further communicates with the phase transfer assembly.

33. A cartridge comprising:
a) a sample collector port comprising a receptacle configured to engage a sample collector, e.g., through a fiction fit or a screw mechanism;
b) a sample chamber, optionally comprising a liquid sample comprising an analyte, communicating with the sample collector port through a fluidic channel, wherein fluid flow between the sample chamber and the sample collector port is optionally regulated by a first valve;
c) a phase transfer compartment comprising a bottom port and a top port, optionally comprising a layer of media for removal of particulates and a desiccant layer, communicating with the sample chamber port through a fluidic channel at the bottom port, wherein fluid flow between the phase transfer compartment and the sample chamber is optionally regulated by a second valve;
c) a solvent chamber, optionally comprising an organic solvent, communicating with the phase transfer compartment through a fluidic channel at the bottom port, wherein fluid flow between the solvent chamber and the phase transfer compartment is optionally regulated by a third valve;
e) a solvent transfer interface port communicating with the solvent chamber through a fluidic channel and configured to engage an instrument comprising a solvent source; and
f) a product port communicating with the phase transfer assembly through a fluidic channel at the top port.

34. The cartridge of claim 33, comprising a base and a cover, wherein the base comprises the fluidic channels communicating between the sample collector port, sample chamber, the phase transfer compartment and the solvent chamber, and a cover comprises the fluidic channel communicating between the product port and the phase transfer chamber.

35. The cartridge of claim 34, wherein the cover comprises sleeves that fit over each of sample chamber, the phase transfer assembly and the solvent chamber.

36. The cartridge of claim 33, wherein the sample chamber and the solvent chamber are configured as pumps comprising a drive to impart positive and/or negative pressure to the chamber compartment.

37. The cartridge of claim 36, wherein the drive comprises a plunger.

38. The cartridge of claim 34, wherein the cover is secured on the base through a snap fit or a friction fit.

39. The cartridge of claim 33, comprising a handle.

40. The cartridge of claim 33, comprising a guide or key to engage a cartridge interface of an instrument.

41. A method comprising:
a) providing a sample comprising a first analyte;
b) loading the sample onto a first phase transfer assembly, wherein the first phase transfer assembly comprises:
i) a first filter layer comprising media configured to filter out particulate matter having a size of at least 0.1 microns, 1 μm, 10 μm, or 100 μm; and
ii) a selective binding layer comprising a desiccant configured to adsorb water from the sample; and
c) eluting the first analyte from the first phase transfer assembly by flowing a non-aqueous solvent through the first phase transfer assembly.

42. The method of claim 41, wherein the first phase transfer assembly further comprises:
iii) a first phase transfer layer comprising media that binds the analyte; and
c) eluting comprises:
i) flowing a first solvent through the first phase transfer assembly to elute unbound material in a first eluate; and
ii) flowing a second solvent through the first phase transfer assembly to extract the first analyte bound to the media therein.

43. The method of claim 42, further comprising:
d) loading the first eluate onto a second phase transfer assembly, where the second phase transfer assembly comprises:
iii) a second phase transfer layer comprising media that binds a second analyte in the first eluate;
e) eluting the second analyte from the second phase transfer assembly by flowing a solvent through the second phase transfer assembly to extract the second analyte bound to the media therein.

44. The method of claim 43, further comprising:
d) loading the first eluted analyte onto a second phase transfer assembly, where in the second phase transfer assembly comprises:

45. The method of claim 43, wherein the sample is loaded at a bottom of the phase transfer assembly and eluted from a top of the phase transfer assembly.
i) a second filter layer comprising particles configured to filter out particulate matter having a size of at least 0.01 microns from the sample; and
ii) a second selective binding layer a desiccant configured to adsorb water from the sample; and
iii) a second phase transfer layer comprising media that binds the second analyte; and
e) eluting the second analyte from the phase transfer assembly with a first organic solvent and the second analyte from the first with a first.

46. The method of claim 42, wherein the phase transfer layer binds the analyte, and extracting the analyte comprises changing the pH of the phase transfer layer so that the analyte no longer binds.

47. The method of claim 43, wherein the sample comprises a first and a second analyte, wherein the media in the phase transfer layer binds the second analyte, and wherein eluting comprises eluting the first analyte with a first organic solvent and eluting the second analyte with a second organic solvent.

48. The method of claim 41, wherein the sample is an aqueous sample comprising a cannabinoid, e.g., tetrahydrocannabinol (“THC”), the first filter layer comprises silica sand (e.g., about 220 micron), the selective binding layer comprises silica gel (e.g., 40-60 microns) and non-aqueous solvent comprises dichloromethane.

49. The method of claim 41, wherein the sample is an aqueous sample comprising cocaine or an opioid, the first filter layer comprises silica sand (e.g., about 220 micron), the selective binding layer comprises silica gel (e.g., 40-60 microns) and non-aqueous solvent comprises a dichloromethane/methanol mixture or dichloromethane/tributylamine mixture.

50. The method of claim 41, further comprising detecting the analyte in the eluate.

51. A method comprising:
a) moving a liquid sample comprising an analyte from a sample collector into a sample chamber;
b) moving the liquid sample from the sample chamber into a phase transfer assembly;
c) moving an organic solvent from a solvent chamber into the phase transfer assembly; and
d) eluting the analyte from the phase transfer assembly with the organic solvent.

52. The method of claim 51, further comprising:
e) moving an organic solvent from a solvent reservoir into the solvent chamber.

53. The method of claim 51, further comprising detecting the eluted analyte with a detector.

54. The method of claim 51, wherein moving a liquid sample comprising an analyte from a sample collector into a sample chamber comprises applying negative pressure.

55. The method of claim 51, wherein moving the liquid sample from the sample chamber into a phase transfer assembly comprises applying positive pressure.

56. The method of claim 51, wherein moving the organic solvent from the solvent chamber into the phase transfer assembly comprises applying positive pressure.

57. The method of claim 51, wherein eluting the analyte from the phase transfer assembly comprises applying positive pressure.

58. The method of claim 51, wherein moving the organic solvent from the solvent reservoir into the solvent chamber comprises applying negative pressure.

59. The method of claim 51, performed by a system of claim 1.
